(19) United States
(12) Patent Application Publication
(10) Pub. No.: US 2012/0303100 A1
(43) Pub. Date: Nov. 29, 2012

(54) Phototherapy Apparatus with Built-In Pressure Sensor

(75) Inventors: Brian Pryor, Newark, DE (US); Sean Xiaolu Wang, Wilmington, DE (US)

(73) Assignee: BWT PROPERTY, INC., Newark, DE (US)

(21) Appl. No.: 13/476,058

(22) Filed: May 21, 2012

Related U.S. Application Data
(60) Provisional application No. 61/490,629, filed on May 27, 2011.

(51) Int. Cl. A61N 5/06

(52) U.S. Cl. 607/90

(57) ABSTRACT

An improved phototherapy apparatus for preventing and treating pressure ulcer and other abnormal skin conditions. The phototherapy apparatus comprises a flexible pad which can be built into or placed on top of a bed. A plurality of light emitting diode (LED) or laser light sources and a plurality of pressure sensors are embedded in the flexible pad. Each pressure sensor is associated with one LED or laser light source for monitoring the pressure endured by a specific body area of the patient. When a pressure above certain level is observed, the corresponding LED or laser light source is turned on to provide phototherapy treatment to the pressured area for preventing and treating pressure ulcer and other abnormal skin conditions.

### REFERENCE TO RELATED APPLICATION

This application claims an invention which was disclosed in Provisional Patent Application No. 61/490,629, filed May 27, 2011, entitled “PHOTOTHERAPY APPARATUS WITH BUILT-IN PRESSURE SENSOR”. The benefit under 35 USC §119(e) of the above mentioned United States Provisional Applications is hereby claimed, and the aforementioned applications are hereby incorporated herein by reference.

### FIELD OF THE INVENTION

This invention generally relates to a phototherapy apparatus and more specifically to a phototherapy apparatus with built-in pressure sensor.

### BACKGROUND

Phototherapy relates to treatment of biological tissues using visible, ultraviolet, and/or infrared lights. The light treatment may be applied solely for sterilization, bio-modulation, and photo-rejuvenation. Alternatively, the treatment may be used in combination with certain photo-sensitive drugs or nutrition supplements. In comparison with laser surgery, the light intensity employed in phototherapy is much lower. Thus the light sources used in phototherapy are not limited to lasers but may include light emitting diodes (LEDs) and/or certain lamps as well. Typical applications of phototherapy include wound healing, cellulite reduction, skin rejuvenation, pain relief, fat reduction and contouring, laser acupuncture, pressure ulcer treatment, etc.

Pressure ulcer or bedsore is a kind of ulcer caused by prolonged pressure or rubbing on vulnerable areas of the body. In its early stage, pressure ulcer appears in the subcutaneous tissue between the skin and bone as pockets of edema. Such dermal damage is hardly observable from the surface of the skin. This places a barrier for using phototherapy to treat these abnormal skin conditions in their early phases, when the phototherapy is most effective.

There thus exists a need for an improved phototherapy apparatus, which is capable of preventing and treating pressure ulcer and other abnormal skin conditions when they are still in their early stages.

### SUMMARY OF THE INVENTION

It is thus the goal of the present invention to provide an improved phototherapy apparatus for preventing and treating pressure ulcer and other abnormal skin conditions. The phototherapy apparatus comprises a flexible pad which can be built into or placed on top of a bed. A plurality of light emitting diode (LED) or laser light sources and a plurality of pressure sensors are embedded in the flexible pad. Each pressure sensor is associated with one LED or laser light source for monitoring the pressure endured by a specific body area of the patient. When a pressure above certain level is observed, the corresponding LED or laser light source is turned on to provide phototherapy treatment to the pressured area for preventing and treating pressure ulcer and other abnormal skin conditions.

### BRIEF DESCRIPTION OF THE FIGURES

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present invention.

FIG. 1 illustrates an exemplary phototherapy pad with built-in pressure sensor.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present invention.

### DETAILED DESCRIPTION

Before describing in detail embodiments that are in accordance with the present invention, it should be observed that the embodiments reside primarily in combinations of method steps and apparatus components related to a phototherapy pad with built-in pressure sensor. Accordingly, the apparatus components and method steps have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus. An element proceeded by “comprises . . . a” does not, without more constraints, preclude the existence of additional identical elements in the process, method, article, or apparatus that comprises the element.

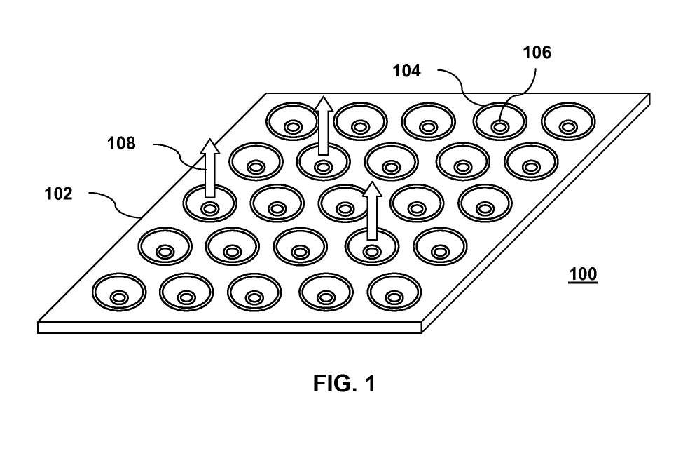

An exemplary embodiment of the phototherapy apparatus is shown in FIG. 1. The phototherapy apparatus 100 comprises a flexible pad 102 with a plurality of embedded light sources 104, which can be light emitting diodes (LEDs) and/or laser diodes. The flexible pad 102 can be built into or placed on top of a bed (not shown). The plurality of light sources 104 are evenly distributed throughout the flexible pad 102 (or embedded in the pad 102 in a predetermined distribution) for producing therapeutic light 108 to treat a patient (not shown) who rests on the bed. A plurality of pressure sensors 106 are also embedded in the flexible pad 102. When the patient rests on the bed, different body areas of the patient will endure different pressures. Each pressure sensor 106 is associated with one light source 104 for monitoring the pressure endured by a specific body area of the patient above the light source and producing a pressure signal. The pressure sensor 106 controls the on/off status of the associated light source 104 based on the measured pressure signal. When a pressure above certain threshold level is observed by a pressure sensor 106, the corresponding LED and/or laser diode light source 104 is turned on to produce therapeutic light 108, which treat the pressured area for preventing and treating pressure ulcer and other abnormal skin conditions.

The therapeutic light produces photochemical reaction in the subject skin tissue, e.g. up-regulation and down-regulation of adenosine triphosphate (ATP), reactive oxygen species, and nitric oxide. The photochemical reaction in turn produces the following therapeutic effects: (i) stimulating white blood cell activity; (ii) accelerating macrophage activity, growth factor secretion and collagen synthesis; (iii) promoting revascularization and micro-circulation; (iv) increasing fibroblast numbers and collagen production; (v) accelerating epithelial cell regeneration and speeding up wound healing; (vi) increasing growth-phase-specific DNA synthesis; (vii) stimulating higher activity in cell proliferation and differentiation; (viii) increasing the intra and inter-molecular hydrogen bonding. All these therapeutic effects combine to prevent the pressured body area from suffering pressure ulcer or provide treatment to pressure ulcer and other abnormal skin conditions when they are still in their early stages.

As an additional feature, the pressure sensor 106 can provide feedback control to the output power of the light source 104 in order to maintain a constant fluence for the therapeutic light 108. The skin tissue is a heterogeneous structure, which has spatial variations in its optical properties. This spatial variation makes the skin tissue a highly scattering optical medium. The scattering loss for the therapeutic light is affected by skin density and skin thickness, which in turn is influenced by the external pressure endured by each body area of the patient. By monitoring this pressure through the pressure sensor 106, the skin density and skin thickness, hence the scattering loss of the area can be estimated from the pressure signal. Via a feedback control loop (not shown), the pressure sensor can control the output power of the light source 104 by adjusting the drive current or voltage of the light source 104 in accordance to the pressure signal such that a constant fluence of therapeutic light is delivered into the skin tissue to obtain an optimum therapeutic result.

The light source 104 may comprise multiple LEDs and/or laser diodes with different output wavelengths, each matching with the absorption band of certain type of chromophores in the skin tissue. For example, light at 810 nm is well absorbed by the hemoglobin and melanin content of the tissue, while light at 980 nm is efficiently absorbed by the water content. The light source may operate in a pulsed mode such that a high peak power is produced to increase the penetration depth of the therapeutic light and/or to trigger nonlinear photochemical reactions yet the average power of the therapeutic light is maintained at low levels to avoid any tissue damage.

In the foregoing specification, specific embodiments of the present invention have been described. However, one of ordinary skill in the art appreciates that various modifications and changes can be made without departing from the scope of the present invention as set forth in the claims below. The numerical values cited in the specific embodiment are illustrative rather than limiting. Accordingly, the specification and figures are to be regarded in an illustrative rather than a restrictive sense, and all such modifications are intended to be included within the scope of present invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

What is claimed is:

1. A phototherapy apparatus for preventing and treating pressure ulcer and other abnormal skin conditions for a patient, the phototherapy apparatus comprising:
a flexible pad for the patient to rest on;
a plurality of light sources embedded in the flexible pad in a predetermined distribution for producing therapeutic light to prevent and treat pressure ulcer and other abnormal skin conditions for the patient; and
a plurality of pressure sensors embedded in the flexible pad, each pressure sensor being associated with a respective light source among the plurality of light sources for monitoring a pressure endured by a body area of the patient above the light source and producing a pressure signal, wherein the pressure sensor controls properties of the light source in accordance to the pressure signal.

2. The phototherapy apparatus of claim 1, wherein said properties of the light source comprises an on-off status.

3. The phototherapy apparatus of claim 1, wherein said properties of the light source comprises an output power.

4. The phototherapy apparatus of claim 1, wherein said plurality of light sources comprise laser diodes.

5. The phototherapy apparatus of claim 1, wherein said plurality of light sources comprise light emitting diodes (LEDs).

6. The phototherapy apparatus of claim 1, wherein said plurality of light sources produce therapeutic light in multiple wavelengths.

7. A method for preventing and treating pressure ulcer and other abnormal skin conditions for a patient, the method comprising the steps of:
providing a flexible pad for the patient to rest on;
providing a plurality of light sources embedded in the flexible pad in a predetermined distribution for producing therapeutic light to prevent and treat pressure ulcer and other abnormal skin conditions for the patient; and
providing a plurality of pressure sensors embedded in the flexible pad, each pressure sensor being associated with a respective light source among the plurality of light sources for monitoring a pressure endured by a body area of the patient above the light source and producing a pressure signal, wherein the pressure sensor controls properties of the light source in accordance to the pressure signal.

8. The method of claim 7, wherein said properties of the light source comprises an on-off status.

9. The method of claim 7, wherein said properties of the light source comprises an output power.

10. The method of claim 7, wherein said plurality of light sources comprise laser diodes.

11. The method of claim 7, wherein said plurality of light sources comprise light emitting diodes (LEDs).

12. The method of claim 7, wherein said plurality of light sources produce therapeutic light in multiple wavelengths.
